(19) United States
(12) Patent Application Publication
(10) Pub. No.: US 2009/0125094 A1
(43) Pub. Date: May 14, 2009

(54) SHEATH FOR INTRAVASCULAR INFLATABLE STRUCTURES, FOR EXAMPLE TO EXPAND STENTS

(75) Inventor: Matthew James Rust, North Vancouver (CA)

Correspondence Address: ANGIOTECH, P.O. BOX 2840, NORTH BEND, WA, 98045, US

(73) Assignee: ANGIOTECH PHARMACEUTICALS, INC., Vancouver, BC (CA)

(21) Appl. No.: 12/266,053

(22) Filed: Nov. 6, 2008

Related U.S. Application Data
(60) Provisional application No. 60/985,856, filed on Nov. 6, 2007.

(51) Int. Cl. A61F 2/84; B23P 11/00

(52) U.S. Cl. 623/1.12; 29/428

(57) ABSTRACT

One or more sheaths limit radial expansion of an expansion structure, for example a multi-balloon catheter structure, while not appreciably limiting bending, facilitating the use of flexible stents in tortuous passages to, for example, perform angioplasty and/or expand stents.

### BACKGROUND

1. Technical Field

The present disclosure is related to in vivo medical devices, and more particularly to inflatable devices for use in expanding stents in bodily vessels, for example vasculature such as arteries.

2. Description of the Related Art

Numerous medical procedures now employ catheters to enter bodily vessels, for example vasculature such as arteries, veins, and or the heart. Such procedures are often percutaneous, providing numerous advantages over more traditional surgery, for example reduced trauma and faster healing. However, catheter procedures are often difficult to perform. Catheter procedures require a care provider (e.g., medical doctor or physician) to guide a catheter or guide wire through the vasculature to a desired position without direct visual feedback. The care provider may receive tactile feedback and/or secondary visual feedback. The secondary visual feedback may come from a camera mounted to the catheter or guide wire. The secondary visual feedback may alternatively come from detection of the catheter or guide wire using medical imaging systems for example ultrasound, computer tomography (CT), realtime magnetic resonance (MR), X-ray, fluoroscopy and/or other radiological apparatus and methods. In either case, the care provider does not have direct visual contact with the catheter or guide wire.

A variety of procedures referred to as angioplasty, widen vasculature and/or remove blockages from vasculature using an inflatable structure such as a balloon positioned at a distal end of a catheter. Often the procedure will include implantation of a structure such as a stent in vasculature to support the vasculature. While some stents are self-expanding, other stents are expanded to contact the wall of the vasculature using a balloon.

The catheter typically includes at least one inflation lumen which allows the balloon to be inflated and deflated and a guide wire lumen allowing the catheter to be guided in the bodily vessel. Balloons are typically made of flexible but inelastic materials, which do not appreciably stretch even when subjected to the high pressures (e.g., 10-30 ATM) commonly employed to inflate such balloons). Hence, the balloon is often folded when in an uninflated state or configuration to provide a relatively small cross sectional diameter. This allows the balloon to fit in the vasculature and/or to fit in the catheter. Rather than stretching, the balloon unfolds in response to inflation, to provide a relatively large cross sectional diameter when in an inflated state or configuration. This allows the balloon to physically engage the walls of the vasculature and/or expand the stent to physically engage the walls of the vasculature.

Typically, catheters must travel a tortuous path through the vasculature. Flexible catheters and/or guide wires have been developed to facilitate such travel. Flexible stents are also now available, which facilitate travel through the vasculature. However, the flexibility of such stents tends to be limited by the flexibility of the inflation balloon. Consequently, numerous short stents may be employed to provide support to an appreciable length of the vasculature. However, each stent requires a respective balloon which increases the complexity of the catheter procedure, which as previously noted is a difficult procedure to perform. Using multiple short stents also leads to discontinuities of vasculature.

Improvements in devices and methods associated with catheter based medical procedures are highly desirable.

### BRIEF SUMMARY

Applicant has recognized that portions of stent expanding inflatable structures such as balloons may extend beyond one or more ends of the stent. As such, a stent is not able to limit a radial expansion of such balloons over the protruding portion. Such may adversely allow those portions of the inflatable structures to over expand.

At least one embodiment may be summarized as an intravascular catheter device to expand a stent, including a catheter having at least one lumen; and a plurality of inflatable chambers physically coupled to the catheter to move through vasculature therewith, the inflatable chambers fluidly coupled with the at least one lumen of the catheter; and a sheath disposed radially outward of the inflatable chambers and radially inwardly of the stent to radially constrain the inflatable chambers when the inflatable chambers are in the relatively more inflated state without appreciably limiting bending of the inflatable chambers.

The inflatable chambers may be formed by at least one balloon and the sheath may be formed of a same type of material as the balloon. An outer diameter of the sheath may be sized to be closely received by an inner diameter of the stent. The plurality of inflatable chambers may be formed by respective ones of a plurality of balloons. The plurality of inflatable chambers may be formed by respective narrowed portions of at least one balloon. The narrowed portions may be formed by respective ones of a number of folds in the at least one balloon. The narrowed portions may be formed by respective ones of a number of necked portions in the at least one balloon.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a cross sectional view of a bodily vessel with a flexible stent deployed therein according to one illustrated embodiment.

FIG. 2A is an isometric view of a sheath according to one illustrated embodiment, which may received between a stent and a plurality of parallel inflatable balloons, the sheath extending along substantially along a length of the inflatable balloons to limit a radial expansion of the inflatable balloons.

FIG. 2B is a front view of the sheath of and the plurality of balloons of FIG. 2A in an inflated configuration, the expansion of the inflatable balloons radially limited by the sheath.

FIG. 2C is a front view of the sheath and the plurality of inflatable balloons of FIG. 2A in an uninflated configuration, showing a diameter to which a radial expansion of the balloons is limited to by the sheath.

FIG. 2D is an isometric view of the sheath of FIG. 2A, illustrating an interior of passage thereof.

FIG. 3 is an isometric view of a sheath according to one illustrated embodiment, which may received between a stent and a plurality of serial inflatable balloons, the sheath extending along substantially along a length of the inflatable balloons to limit a radial expansion of the inflatable balloons.

FIG. 4 is an isometric view of a pair of sheaths according to one illustrated embodiment, disposed about respective portions of a plurality of inflatable structures which extend or protrude beyond the ends of a stent.

FIG. 5 is an isometric view of a sheath according to another illustrated embodiment.

FIG. 6 is an isometric diagram of a according to a further illustrated embodiment.

FIG. 7 is flow diagram showing a method of producing a medical device according to one illustrated embodiment.

FIG. 8 is a flow diagram showing a method of producing a medical device according to one illustrated embodiment.

FIG. 9 is a flow diagram showing a method of producing a medical device according to another illustrated embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with stents, catheters, balloons, fluid delivery subsystems and/or image based or other location determination subsystems have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

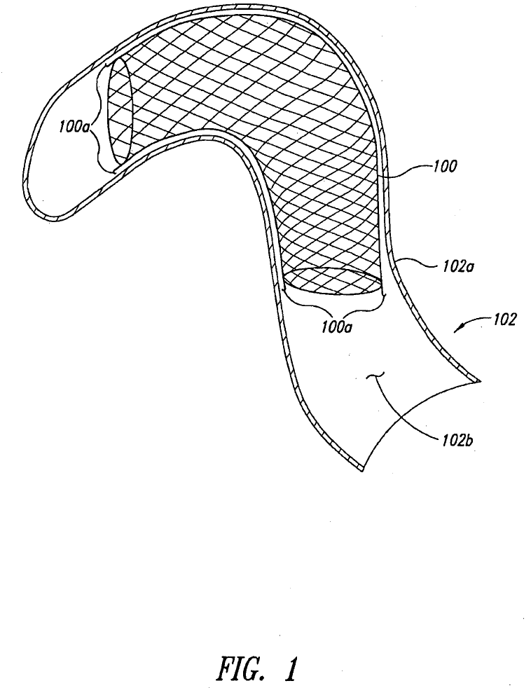

FIG. 1 shows a flexible stent 100 in a portion of a bodily vessel 102 according to one illustrated embodiment.

The bodily vessel 102 may take a variety of forms, for example an artery, vein, or other vasculature. A wall 102a of the vessel 102 defines at least one passage or lumen 102b. The lumen 102a of the bodily vessel 102 may present a torturous path through which the stent 100 must travel to reach a desired position.

The stent 100 may take a variety of forms, for example a balloon expandable stent. The balloon expandable stents 100 may have various forms, for example tube, wire, sheet or ribbon stents. Balloon expandable stents 100 may be formed of a variety of materials, for example stainless steel, tantalum, nitinol, platinum iridium, polymers, niobium alloy, or cobalt alloys. Balloon expandable stents 100 may be formed by way of a variety of processes, for example laser cutting, photochemical etching, electronic discharge machining, water jet cutting, braiding or knitting. Balloon expandable stents may also have a variety of geometries, for example coil, helical spiral, woven, individual rings unconnected, open cell, closed cell, peak-to-peak, peak-to-valley and hybrids. Helical spirals geometries may, for example, be integral, have an axial spine, have no or minimal connections of periodic peak-to-peak connections. Woven geometries may, for example, be braided or knitted (where knitted structures may include the interloping of one or more continuous threads). Closed cell geometries may, for example, include regular peak-to-peak connections, with non-flexible connectors, flexible connectors or a combination of flexible and non-flexible connectors.

The stent 100 may include one or more structures such as hooks 100a that anchor, implant or otherwise secure the stent 100 to the wall 102a of the bodily vessel 102 at a desired position. The stent 100 may travel through the lumen 102b in a compressed or non-expanded configuration, for example being advanced via a catheter. The stent 100 may be expanded to physically engage the wall 102a at the desired position, for example using an expansion structure formed from two or more inflatable structures. As discussed in more detail below, the inflatable structures may take the form of one or more balloons with one or more inflatable chambers.

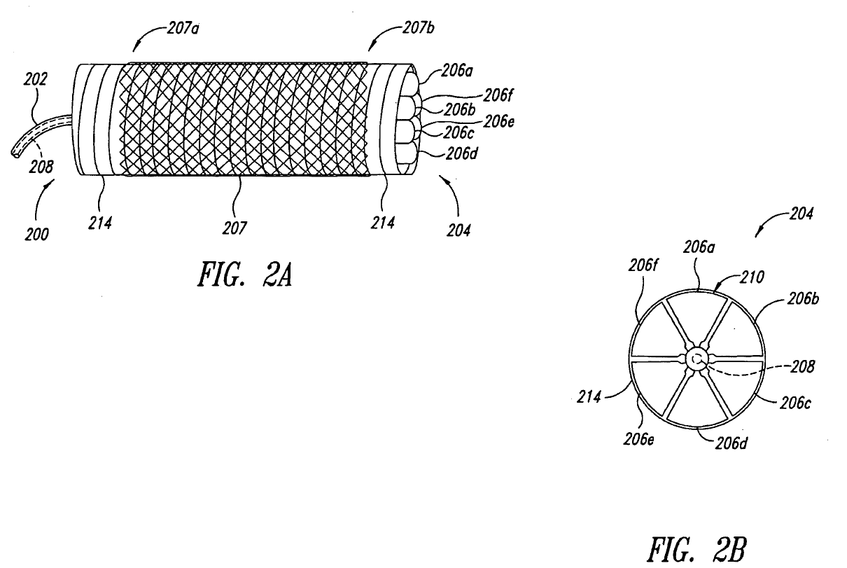

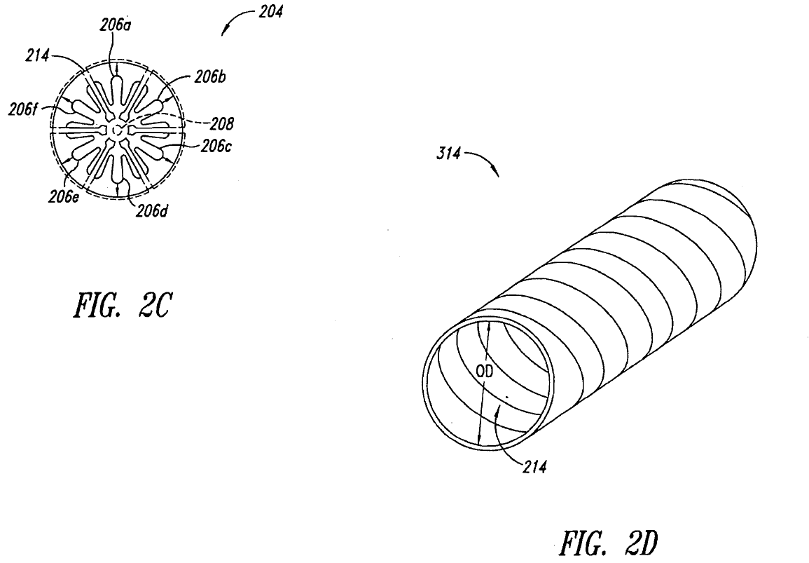

FIG. 2A shows a catheter device 200, according to one illustrated embodiment.

The catheter device 200 includes a flexible elongated member 202 and an expansion structure 204 positioned at least proximate one end of the flexible elongated member 202. The expansion structure 204 includes a plurality of inflatable structures such as balloons 206a-206f (collectively 206). The balloons 206 may extend generally parallel to one another. In at least some embodiments where the balloons 206 are to be used with one or more stents 207, the balloons 206 may have a length that is longer than a length of the stent 207. Consequently, the balloons may extend or protrude beyond one or more ends 207a, 207b of the stent 207.

The balloons 206 include interiors or chambers which may be fluidly communicatively coupled to one or more lumens 208 of the flexible elongated member 202. As illustrated in FIGS. 2B and 2C, the balloons 206 may be inflated from a relatively uninflated state or configuration (FIG. 2C) into a relatively inflated state or configuration (FIG. 2B) by providing fluid to the interior of the balloons via the lumen 208.

In some embodiments, the balloons 206 are made of inelastic material and hence do not appreciably stretch when unfolding even under relatively high pressures associated with balloons that expand stents. Thus, one or more of the balloons 206 may be in a folded configuration, such as that illustrated in FIG. 2C, which provides a cross section having a relatively small or reduced diameter for travel through the bodily vessel to the desired position. In response to inflation, the balloon 206 unfolds (as illustrated by arrows) into the relatively inflated state or configuration, for example as illustrated by broken lines in FIG. 2C.

In some embodiments, the balloons 206 may be elastic, appreciably stretching when inflated. In such embodiments the balloons 206 may or may not be folded since the profile or cross-section of the balloons 206 will be relatively small when uninflated versus when inflated. The balloons 206 may also be deflated, allowing removal of the balloons 206, for example after the balloons 206 have expanded a stent.

Individual balloons 206 when expanded or unfolded may have a generally circular undeformed cross section. Such balloons are advantageously easy and inexpensive to manufacture as compared to more complex geometries. Where two or more of the balloons 206 are grouped together, neighboring balloons 206 may physically interfere with one another as the balloons 206 inflate and unfold. The adjacent portions of the balloons 206 are shown with a space therebetween in order to illustrate the individual balloons. In actual use, the adjacent portions of the balloons 206 would be physically touching one another, physical interfering with expansion. Thus, the balloons 206 which might otherwise have a circular cross section may have a wedge or pie shape cross section, best illustrated in FIG. 2B. Notably, an outer perimeter portion 210 (only one called out in FIG. 2B) of each of the balloons 206 is approximately arcuate, and the group of balloons 206 have a substantially circular cross section.

The expansion structure 204 may be advanced through the bodily vessel 102 (FIG. 1) using the flexible elongated member 202 of the catheter device 200. When in a desired position, fluid can be provided via one or more lumens 208 of the flexible elongated member 202 to inflate the balloons 206, causing the balloons to unfold into the relatively inflated state or configuration. Thus the balloons 206 move from a relatively less inflated state or configuration, illustrated in FIG. 2C, to a relatively more inflated state configuration, illustrated in FIG. 2B.

A sheath 214 may limit a radial expansion of one or more portions of the balloons 206. For example, the sheath 214 may radially limit expansion of any portions that extend or protrude beyond the ends 207a, 207b of the stent 207. Typically, the stent 207 will be sufficiently stiff to limit radial expansion of the portions of the inflatable balloons 206 about which the stent 207 is disposed. However, the sheath 214 may extend under the stent 207 to either limit radial expansion or simply for convenience since the sheath 214 will typically extend beyond the ends 207a, 207b of the stent 207.

As best seen in FIG. 2D, the sheath 214 has a passage 216 sized to receive the balloons 206 of the expansion structure 204. The sheath 214 may be formed of one or more helically wound members, which allows the sheath 214 to bend in order to follow a torturous path. An inner diameter OD of the sheath 214 may limit the expansion of the balloons 206 such that the inner diameter of the sheath 214 defines a maximum outer diameter of the group of balloons 206. The sheath 214 may also be made as a woven structure, a corrugated structure or any other structure that limits radial expansion without severely limiting bending flexibility (and as such may include nonwoven structures such as those in which the material(s) selected themselves provide bending flexibility). For example, the sheath 214 may not limit bending of the balloons 206 by greater than approximately 10 percent more than the balloons 206 without the sheath 214. The closer sheath 214 comes to not limiting the bending (e.g., 5%, 2%, 1% or equal or less than 0%) of the balloons 206, the better, although such may have to be balanced against the ability of the sheath 214 to retain radial expansion of the balloons 206 and/or thickness of the sheath 214 and hence overall cross sectional diameter of the expansion structure.

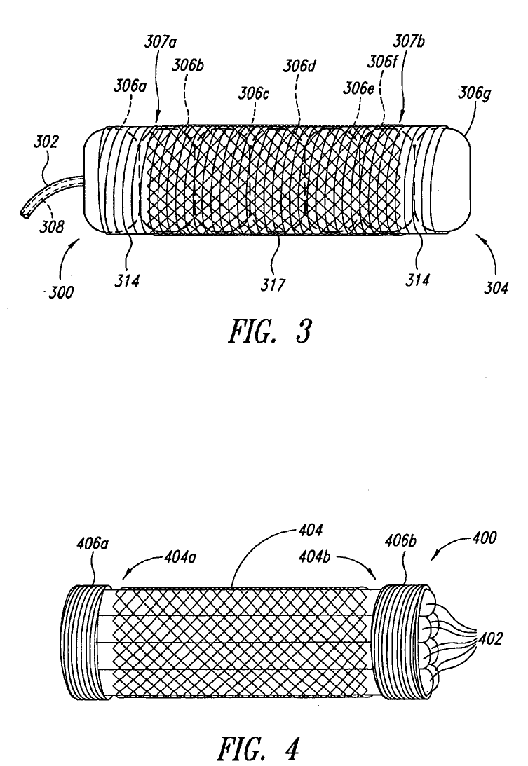

FIG. 3 shows a catheter device 300, according to one illustrated embodiment.

The catheter device 300 includes a flexible elongated member 302 and an expansion structure 304 positioned at least proximate one end of the flexible elongated member 302. The expansion structure 304 includes a plurality of inflatable structures or chambers formed by bends or necked portions, for example inflatable structures such as balloons 306a-306g (collectively 306). The balloons 306 may extend generally serially with respect to one another. In at least some embodiments where the balloons 306 are to be used with one or more stents 307, the serial arrangement of the balloons 306 may have a cumulative length that is longer than a length of the stent 307. Consequently, the balloons may extend or protrude beyond one or more ends 307a, 307b of the stent 307.

The balloons 306 include interiors or chambers which may be fluidly communicatively coupled to one or more lumens 308 of the flexible elongated member 302. The balloons 306 may be inflated from a relatively uninflated state or configuration into a relatively inflated state or configuration by providing fluid to the interior of the balloons via the lumen 308.

In some embodiments, the balloons 306 are made of inelastic material and hence do not appreciably stretch when unfolding even under relatively high pressures associated with balloons that expand stents. Thus, one or more of the balloons 306 may be in a folded configuration, which provides a cross section having a relatively small or reduced diameter for travel through the bodily vessel to the desired position. In response to inflation, the balloon 306 unfolds into the relatively inflated state or configuration.

In some embodiments, the balloons 306 may be elastic, appreciably stretching when inflated. In such embodiments the balloons 306 may or may not be folded since the profile or cross-section of the balloons 306 will be relatively small when uninflated versus when inflated. The balloons 306 may also be deflated, allowing removal of the balloons 306, for example after the balloons 306 have expanded a stent.

Individual balloons 306 when expanded or unfolded may have a generally circular undeformed cross section or spherical shape. Such balloons are advantageously easy and inexpensive to manufacture as compared to more complex geometries. Where two or more of the balloons 306 are grouped together, neighboring balloons 306 may physically interfere with one another as the balloons 306 inflate and unfold. The adjacent portions of the balloons 306 are shown with a space therebetween in order to illustrate the individual balloons. In actual use, the adjacent portions of the balloons 306 would be physically touching one another, physical interfering with expansion. Thus, the balloons 306 which might otherwise have a circular cross section may have a more oval or D-shaped cross section, as illustrated in FIG. 3. Notably, an outer perimeter portion of each of the balloons 306 in a cross section taken perpendicular to a longitudinal axis of the structure is approximately circular. Thus, the balloons 306 may apply a relatively even force about the circumference of the stent 307

The expansion structure 304 may be advanced through the bodily vessel 102 (FIG. 1) using the flexible elongated member 302 of the catheter device 300. When in a desired position, fluid can be provided via one or more lumens 308 of the flexible elongated member 302 to inflate the balloons 306, causing the balloons to unfold into the relatively inflated state or configuration.

A sheath 314 may limit a radial expansion of one or more portions of the balloons 306. For example, the sheath 314 my radially limit expansion of any portions that extend or protrude beyond the ends 307a, 307b of the stent 307. Typically, the stent 307 will be sufficiently stiff to limit radial expansion of the portions of the inflatable balloons 306 about which the stent 307 is disposed. However, the sheath 314 may extend under the stent 307 to either limit radial expansion or simply for convenience since the sheath 314 will typically extend beyond the ends 307a, 307b of the stent 307.

As previously described, the sheath 314 may have a passage sized to receive the balloons 306 of the expansion structure 304. The sheath 314 may be formed of one or more helically wound members, which allows the sheath 314 to bend in order to follow a torturous path. An inner diameter OD of the sheath 314 may limit the expansion of the balloons 306 such that the inner diameter of the sheath 314 defines a maximum outer diameter of any of the balloons 306 that the sheath 314 physically contacts. The sheath 314 may also be made as a woven structure, a corrugated structure or any other structure that limits radial expansion without severely limiting bending flexibility. For example, the sheath 314 may not limit bending of the balloons 306 by greater than approximately 10 percent more than the balloons 306 without the sheath 314. The closer sheath 314 comes to not limiting the bending (e.g., 5%, 2%, 1% or equal or less than 0%) of the balloons 306, the better, although such may have to be balanced against the ability of the sheath 314 to retain radial expansion of the balloons 306 and/or thickness of the sheath 314 and hence overall cross sectional diameter of the expansion structure.

FIG. 4 shows an expansion structure 400 including a plurality of inflatable structures 402, received in a stent 404, and a pair of sheaths 406a, 406b (collectively 406) to radially limit expansion of the inflatable structures, without substantially limiting bending.

The sheaths 406 are radially disposed about respective portions of the inflatable structures 402 that extend from opposed ends 404a, 404b of the stent 404. The sheaths 406 do not extend about or otherwise engage portions of the inflatable structures extending between the opposed ends 404a, 404b of the stent 404, since the stent may sufficiently resist radial expansion of the inflatable structures 402 in such a region. For example, the ends of the inflatable structures 402 that protrude or extend beyond the ends 404a, 400b of the stent 404 may be wrapped with an inelastic thread or received within a short tubular member.

While illustrated as a plurality of elongated, generally parallel structures, in some embodiments the inflatable structures 402 may take the form of a plurality of serially aligned or couple structures, for instance resembling beads on a string. In other embodiments, a sheath may extend over the entire length of the inflatable structures 402, including that portion that is between the opposed ends 404a, 404b of the stent 404.

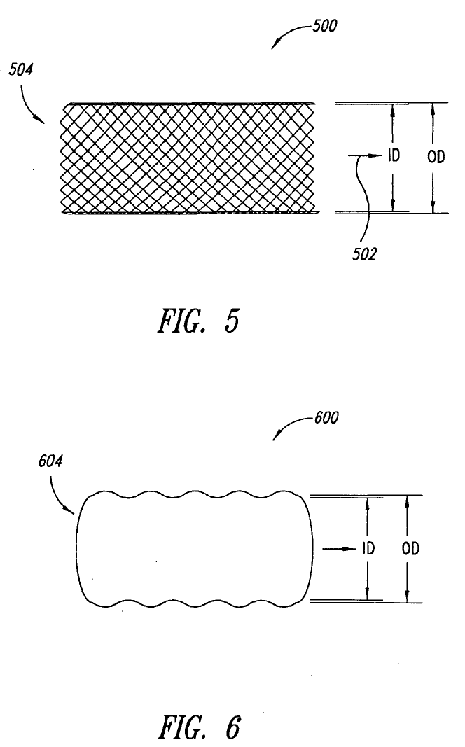

FIG. 5 shows a sheath 500 according to another illustrated embodiment.

The sheath 500 is formed of a woven material. The material may take a variety of forms including polymer based materials, natural materials such as silk, or even metal. The woven structure allows the sheath 500 to bend about a longitudinal axis 502, while limiting radial expansion of one or more balloons received in a passage 504 of the sheath 500. The sheath 500 has an inner diameter ID sized to receive the balloon(s) and to limit expansion of the balloon(s) to a desired diameter. The sheath 500 has an outer diameter OD sized to be closely received by a stent. The stent may be retained on the sheath 500 via a frictional fit. Alternatively, or additionally, the stent may be retained on the sheath 500 via a releasable mechanism and/or releasable adhesive.

FIG. 6 shows a sheath 600 according to a further illustrated embodiment.

The sheath 600 includes a corrugated or bellows-like structure. The corrugated or bellows-like structure allows the sheath 600 to bend about a longitudinal axis 602, while limiting radial expansion of one or more balloons received in a passage 604 of the sheath 600. The sheath 600 has an inner diameter ID sized to receive the balloon(s) and to limit expansion of the balloon(s) to a desired diameter. The sheath 600 has an outer diameter OD sized to be closely received by a stent. The stent may be retained on the sheath 600 via a frictional fit. Alternatively, or additionally, the stent may be retained on the sheath 600 via a releasable mechanism and/or releasable adhesive. The sheath 600 may be formed from a variety of materials, for example polymer based materials, natural materials, or metal materials.

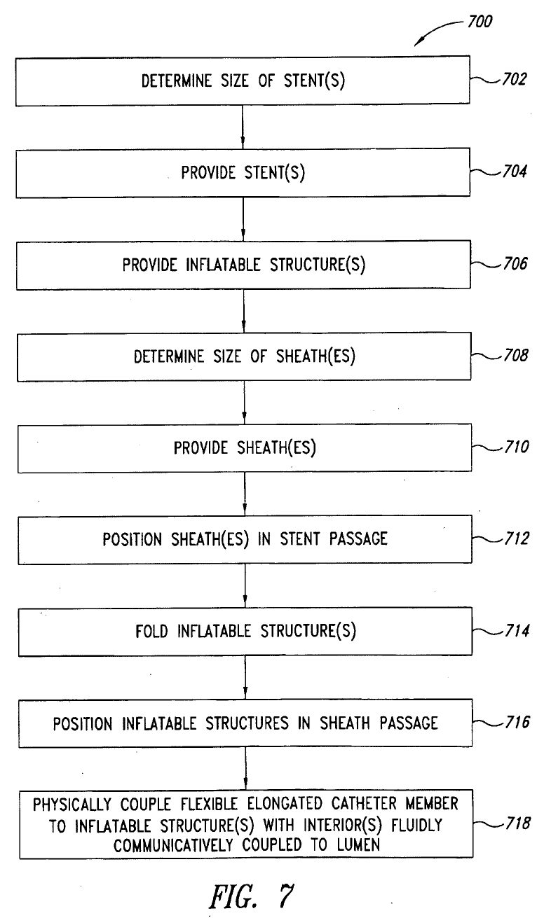

FIG. 7 shows a method 700 of producing a medical device according to one illustrated embodiment.

At 702, a stent size is determined, such that the stent will be sized to be received intravascularly. At 704, one or more appropriately sized stents are provided. Each stents has a pair of opposed ends and a passage that extends therethrough.

At 706, one or ore inflatable structures are provided. Such may include determining an appropriate diameter and/or length, and selecting one or more suitably sized inflatable structures. The diameter may be based on the radial size of the stent, while the length may be based on a length of the stent(s).

At 708, a sheath size is determined. As previously noted, the sheath limits the radial expansion of the inflatable structures, thus an appropriate inner diameter and/or length should be determined. At 710, one or more sheathes of the determined size are provided. At 712, the sheath is positioned in the passage of the stent(s).

At 714, the inflatable structures are folded. At 716, one or more inflatable structure(s) are positioned in the sheath passage and/or the stent passage. At least a portion of at least one of the inflatable structures may protrude from at least one of the ends of the stent. The sheath should be disposed about at least any portions of the inflatable structure that protrude beyond the ends of the stent to radially limit the expansion of those protruding portions.

At 718, a flexible elongated catheter member is physically coupled to the inflatable structures, to guide the inflatable structures through the vasculature along with the stent and sheath(es). At least one lumen of the flexible elongated catheter member is fluidly communicatively coupled to an interior or chamber of the inflatable structures to selectively inflate and deflate the inflatable structures.

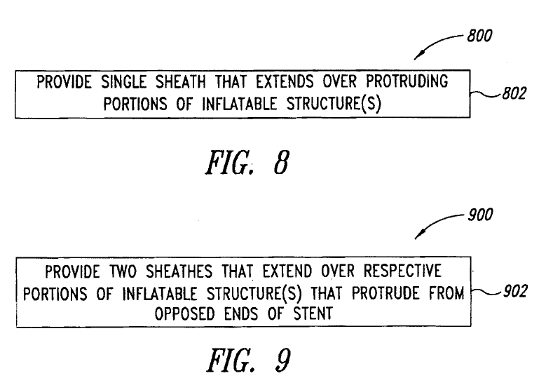

FIG. 8 shows a method 800 of producing a medical device according to one illustrated embodiment.

At 802, a single sheath is provided that extends over the portions of the at least one inflatable structure that protrude from both of the ends of the pair of opposed ends of the stent.

FIG. 9 shows a method 900 of producing a medical device according to another illustrated embodiment.

At 902, two sheathes are provided such that a first one of the sheathes extends over a first one of the portions of the at least one inflatable structure that protrudes from a first one of the ends of the pair of opposed ends of the stent and a second one of the sheathes extends over a second one of the portions of the at least one inflatable structure that protrudes from a second one of the ends of the pair of opposed ends of the stent.

The above description of illustrated embodiments, including what is described in the Abstract, is not intended to be exhaustive or to limit the embodiments to the precise forms disclosed.

Some suitable materials for the various inflatable structures or balloons described herein include polyethylene (PET), flexible polyvinylchloride (PVC) cross-linked polyethylene, non-cross-linked polyolefin, polyurethane, polyetheretherketone (PEEK), silicone, polytetrafluoroethylene (PTFE) or various thermo plastic elastomers. Some embodiments may even employ a thin metal to form the balloons. Some suitable materials for the various flexible elongated members described herein include may include PEEK or silicone. In some embodiments, the balloons and flexible member may be formed of the same materials.

The various embodiments described above can be combined to provide further embodiments. To the extent that they are not inconsistent with the specific teachings and definitions herein, all of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ systems, circuits and concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An intravascular catheter device to expand a stent, the intravascular article comprising:
a catheter having at least one lumen; and
a plurality of inflatable chambers physically coupled to the catheter to move through vasculature therewith, the inflatable chambers fluidly coupled with the at least one lumen of the catheter and inflatable from a relatively less inflated state when moved through the vasculature to a relatively more inflated state when at a desired position in the vasculature; and
at least one sheath disposed radially outward of at least some of the inflatable chambers and radially inwardly with respect to a perimeter of the stent to radially constrain at least some of the inflatable chambers when the inflatable chambers are in the relatively more inflated state without appreciably limiting bending of the inflatable chambers.

2. The intravascular catheter device of claim 1 wherein the inflatable chambers are formed by at least one balloon and the sheath is formed of a same type of material as the balloon.

3. The intravascular catheter device of claim 1 wherein an outer diameter of the sheath is closely received by an inner diameter of the stent.

4. The intravascular catheter device of claim 1 wherein the plurality of inflatable chambers are formed by respective ones of a plurality of balloons.

5. The intravascular catheter device of claim 1 wherein the plurality of inflatable chambers are formed by respective narrowed portions of at least one balloon.

6. The intravascular catheter device of claim 5 wherein the narrowed portions are formed by respective ones of a number of folds in the at least one balloon.

7. The intravascular catheter device of claim 5 wherein the narrowed portions are formed by respective ones of a number of necked portions in the at least one balloon.

8. The intravascular catheter device of claim 1, wherein the structure of the sheath is selected from the class consisting of helical, woven, and corrugated.

9. The intravascular catheter device of claim 1 wherein the sheath does not limit bending of the inflatable chambers by greater than approximately 10 percent more than the inflatable chambers without the sheath.

10. The intravascular catheter device of claim 1 wherein the sheath engages at least one portion of the inflatable chambers that extends longitudinally beyond an end of the stent.

11. The intravascular catheter device of claim 10 wherein the sheath does not engage a portion of the inflatable chambers that extends longitudinally between a pair of opposed ends of the stent.

12. An article of manufacture for use with a stent and an expansion structure, the article comprising:
a sheath having an outer diameter sized to be closely received by the stent and an inner diameter sized to receive an expansion structure and to limit a radial expansion of the expansion structure to a defined diameter, the sheath not appreciably limiting bending of the expansion structure.

13. The article of claim 12 wherein the sheath does not limit bending of the expansions structure by greater than approximately 10 percent more than the expansion structure without the sheath.

14. The intravascular catheter device of claim 12 wherein the structure of the sheath is selected from the class consisting of helical, woven, and corrugated.

15. A method of producing a medical device, the method comprising:
providing at least one stent having a pair of opposed ends and a passage that extends therethrough;
providing at least one inflatable structure received by the passage of the at least one stent, at least a portion of the at least one inflatable structure protruding from at least one of the ends of the stent; and
providing at least one sheath over at least one portion of the at least one inflatable structure that protrudes from at least one of the ends of the stent.

16. The method of claim 15, further comprising:
physically coupling a flexible elongated catheter member having at least one lumen to the at least one inflatable structure and fluidly communicatively coupling to an interior of the at least one inflatable structure to the at least one lumen.

17. The method of claim 15, further comprising:
folding the at least one inflatable structure to be received by the passage of the at least one stent.

18. The method of claim 15 wherein providing at least one sheath over at least one portion of the at least one inflatable structure that protrudes from at least one of the ends of the stent includes providing a single sheath that extends over the portions of the at least one inflatable structure that protrude from both of the ends of the pair of opposed ends of the stent.

19. The method of claim 15 wherein providing at least one sheath over at least one portion of the at least one inflatable structure that protrudes from at least one of the ends of the stent includes providing two sheathes such that a first one of the sheathes extends over a first one of the portions of the at least one inflatable structure that protrudes from a first one of the ends of the pair of opposed ends of the stent and a second one of the sheathes extends over a second one of the portions of the at least one inflatable structure that protrudes from a second one of the ends of the pair of opposed ends of the stent.

20. The method of claim 15 wherein providing at least one stent having a pair of opposed ends and a passage that extends therethrough includes providing a first stent that is sized to be received intravascularly.
